(19) United States
(12) Patent Application Publication
(10) Pub. No.: US 2024/0167076 A1
(43) Pub. Date: May 23, 2024

(54) SELECTIVE ENRICHMENT

(71) Applicant: Harbinger Health, Inc., Cambridge, MA (US)

(72) Inventor: Anthony P. Shuber, Northbridge, MA (US)

(21) Appl. No.: 18/212,371

(22) Filed: Jun. 21, 2023

Related U.S. Application Data
(63) Continuation of application No. 16/987,601, filed on Aug. 7, 2020, now abandoned.
(60) Provisional application No. 62/884,498, filed on Aug. 8, 2019.

(51) Int. Cl. C12Q 1/6806; C12Q 1/6848

(52) U.S. Cl. CPC C12Q 1/6806; C12Q 1/6848

(57) ABSTRACT

Provided herein are methods of detecting nucleic acids. The nucleic acid of interest may be detected by using selective enrichment. At least two Cas endonuclease complexes are introduced to a sample comprising nucleic acid. The Cas endonuclease complexes comprise guide RNAs and Cas endonuclease. The Cas endonuclease complexes attach to a target nucleic acid, thereby protecting the target of interest while unprotected nucleic acid in the sample is degraded, e.g., by exonuclease digestion. Linkers, when added to the sample, will attach to the ends of the target nucleic acid previously protected by the Cas endonuclease and will not attach to the degraded, unprotected nucleic acid in the sample. The target nucleic acid and linkers are then detected.

This application claims the benefit of U.S. Provisional Application 62/884,498 filed on Aug. 8, 2019, the contents of which are hereby incorporated by reference in their entirety.

### TECHNICAL FIELD

The invention generally relates to detection of nucleic acids.

### BACKGROUND

Disease diagnosis and pathogen identification can be difficult, especially if the diagnostic targets are not present in abundance in a sample. For example, it has become possible to detect circulating tumor DNA in blood. However, the difficulty is that tumor DNA fragments are not present in blood in amounts sufficient for straightforward detection with conventional measures, such as PCR. Similarly, pathogens in complex biological samples are often difficult to find in numbers sufficient for identification.

Detection of DNA or RNA is a common means for identifying pathogens. Detection and identification of the relevant DNA or RNA is difficult because the targets are present in an abundance of non-target material. For example, because PCR is stochastic and exponential, if one fails to amplify a relevant target in the first or second round of amplification it is very unlikely that target will ever be found. Similarly, probe hybridization is difficult in a sea of irrelevant material. Finally, the ability to multiplex standard PCR or probe based assays is limited, making it difficult to detect more than one pathogen in a sample.

### SUMMARY

The invention provides methods of detecting nucleic acid in a heterogenous population of nucleic acids by selectively degrading non-target nucleic acids, making detection of the target more likely. Methods of the invention are useful to detect nucleic acid from a pathogen, to characterize a microbiome of an organism, or to perform metagenomic detection of species in a sample. Detection involves a form of negative enrichment in which target nucleic acid is protected and a selective enzymatic digestion of unprotected DNA or RNA is performed. Then, protective groups are removed and linkers are added to enable capture, amplification, and/or sequencing of the targets. In a preferred embodiment, target DNA or RNA is protected using Cas/Ribonucleicprotein (RNP) complexes. Then, when the sample is exposed to a degradative enzyme, for example an exonuclease, unprotected ends are digested (partially or completely), making those ends unavailable for attachment of a linker. Once the Cas/RNP complexes are removed, linkers are added to the protected regions. The linkers can then be used as substrates for PCR primers, for hybridization probes, or for immediate insertion into a third generation sequencing process. Because the nucleic acid of interest has been isolated, simply detecting the presence of the target nucleic acid confirms the presence of, for example, the relevant microbe or pathogen in a subject or sample. Thus, the invention provides methods for rapidly and simply detecting a pathogen in a complex sample, regardless of the presence of nucleic acids from other sources.

Because the methods of the invention provide a simple way to isolate target nucleic acids from a population, they have several advantages over previous methods of target identification. First, the methods are not constrained by the size of the target and thus are able to detect nucleic acids of 10 kb or more. Thus, methods of the invention are useful for preparation of long fragments for third generation sequencing platforms, such as those used by Oxford Nanopore and Pacific Biosciences. In addition, because irrelevant nucleic acids are degraded, the methods are highly sensitive, allowing detection of targets that are present in the population at very low abundance.

The features described above make the methods of the invention useful for detecting microbes, such as pathogenic organisms, in samples from a variety of sources. For example, the methods can be used to detect viral, bacterial, or fungal infections in tissue from a patient or non-human animal. Other applications include detection of microbes in food, environmental water sources, soil, or agricultural materials. Multiplexing versions of the methods allow identification of the microbiome of a bodily tissue or external sample.

The methods are also useful for detecting endogenous nucleic acids in a sample. For example, the methods permit detection of mitochondrial DNA from samples in which nuclear DNA predominates. Alternatively, mutations in chromosomal DNA can be identified.

The methods are also useful for detecting nucleic acid from an infectious agent, such as a virus, as may present in a host. Methods are addressed to challenges by which viral nucleic acid may be difficult to detect among abundant host DNA.

In an aspect, the invention provides methods of detecting a nucleic acid. The methods include protecting a target nucleic acid in a sample and degrading unprotected nucleic acids. Protection can be mediated by Cas endonuclease complexes. Finally, the methods include detecting the protected nucleic acids. Preferably, the Cas complexes are Cas9 complexes. The Cas complexes that protect the ends of the target nucleic acid may be different from each other, or they may be the same. Preferably, all or nearly all of the unprotected nucleic acids are degraded. Preferably, the protected nucleic acids include the target nucleic acid.

The population of nucleic acids may come from any source. For example, the source may be an organism, such as a human, non-human animal, plant, or other type of organism. The source may be a tissue sample from an animal, such as blood, serum, plasma, skin, conjunctiva, gastrointestinal tract, respiratory tract, vagina, placenta, uterus, oral cavity or nasal cavity. The source may be an environmental source, such as a soil sample or water sample, or a food source, such as a food sample or beverage sample. The nucleic acids may be isolated, purified, or partially purified from a source. Alternatively, nucleic acids may be contained in sample that has not been processed.

The target nucleic acid may be from the genome of a pathogen, such as a virus, bacterium, or fungus. The nucleic acids may come from an organism, and the target nucleic acid may be foreign to the genome of the organism. For example, the target nucleic acid may be from a pathogen of the organism. The target nucleic acid may be from a virus that infects the organism from which the nucleic acids are obtained. The target may be a viral nucleic acid that has integrated into the genome of the host organism. Additionally or alternatively, the target may be a viral nucleic acid that exists separately from the nucleic acids of the host organism. The nucleic acids may come from an organism, and the target nucleic acid may be native to the organism. For example, the target nucleic acid may be from the nuclear genome, mitochondrial genome, or chloroplast genome of the organism.

The target nucleic acid may have a particular size. For example, the nucleic acid of interest may be between 100 and 10,000 nucleotides in length, or it may be greater than 10,000 nucleotides. The nucleic acid of interest may be larger than any remaining nucleic acids after degradation. Thus, the difference in size between the nucleic acid of interest and the nucleic acid fragments after digestion may facilitate detection of the nucleic acid of interest.

The Cas complexes include a Cas endonuclease and a guide RNA. The Cas endonuclease may include any Cas endonuclease. For example, the Cas endonuclease may be Cas9, Cas13, Cpf1, C2c1, C2c3, C2c2, CasX, or CasY, including modified versions of Cas9, Cas13, Cpf1, C2c1, C2c3, C2c2, CasX, or CasY in which the amino acid sequence has been altered. The Cas endonuclease is catalytically inactive. For example, the Cas endonuclease may be Streptococcus pyogenes Cas9 that has a D10A and/or a R1335K mutation, Acidaminococcus sp. BV3L6 Cpf1 that has a D908 mutation, or Lachnospiraceae bacterium ND2006 that has a D832 mutation.

The guide RNAs may be any guide RNA that functions with a Cas endonuclease. Individual guide RNAs may include a separate crRNA molecule and tracrRNA molecule, or individual guide RNAs may be single molecules that include both crRNA and tracrRNA sequences.

Protection of the target nucleic acid may include the binding of the Cas complexes to one or both ends. The Cas complexes that bind to the ends of the target nucleic acid may be catalytically inactive. Protection of the ends of the target nucleic acid may include cleavage of the target nucleic acids at one or both ends.

Degradation of unprotected nucleic acids may include digestion with an exonuclease, such as exonuclease I, exonuclease II, exonuclease III, exonuclease IV, exonuclease V, exonuclease VI, exonuclease VII, or exonuclease VIII.

The target nucleic acid may be detected by any suitable means, such as by hybridization, spectrophotometry, sequencing, electrophoresis, amplification, fluorescence detection, or chromatography.

In another aspect, the invention provides methods of detecting a microbe in which the methods include the following steps: protecting the ends of a target nucleic acid from a genome of a microbe in a sample using a pair of Cas complexes; degrading unprotected nucleic acids; and detecting the protected nucleic acid, which indicates the presence of the microbe in the sample. Preferably, the Cas complexes are Cas9 complexes. The Cas complexes in a pair may be different from each other, or they may be the same. Preferably, all or nearly all of the unprotected nucleic acids are degraded. Preferably, the protected nucleic acids include the target nucleic acid.

The methods may include detecting multiple microbes in a sample. For example, the methods may include determining the microbiome of a sample. In such methods, multiple target nucleic acids are detected using multiple pairs of Cas complexes. For example, the methods may include sets of Cas complexes that include at least 1000 pairs. When multiple microbes are detected in a sample, the methods may include determining the relative abundance of different microbes in the sample. The methods may include counting the different target nucleic acids to determine the relative abundance of microbes in the sample. One or more of the microbes in the sample may be pathogens, such as a viruses, bacteria, or fungi.

The sample may come from any source. For example, the source may be an organism, such as a human, non-human animal, plant, or other type of organism. The source may be a tissue sample from an animal, such as blood, serum, plasma, skin, conjunctiva, gastrointestinal tract, respiratory tract, vagina, placenta, uterus, oral cavity or nasal cavity. The source may be an environmental source, such as a soil sample or water sample, or a food source, such as a food sample or beverage sample. The sample may comprise nucleic acids that have been isolated, purified, or partially purified from a source. Alternatively, the sample may not have been processed.

The target nucleic acids may have a particular size. For example, the target nucleic acids may be between 100 and 10,000 nucleotides in length, or it may be greater than 10,000 nucleotides in length.

The Cas complexes include a Cas endonuclease and a guide RNA. The Cas endonuclease may include any Cas endonuclease. For example, the Cas endonuclease may be Cas9, Cpf1, C2c1, C2c3, C2c2, CasX, or CasY, including modified versions of Cas9, Cpf1, C2c1, C2c3, C2c2, CasX, or CasY in which the amino acid sequence has been altered. The Cas endonuclease is catalytically inactive. For example, the Cas endonuclease may be Streptococcus pyogenes Cas9 that has a D10A and/or a R1335K mutation, Acidaminococcus sp. BV3L6 Cpf1 that has a D908 mutation, or Lachnospiraceae bacterium ND2006 that has a D832 mutation.

The guide RNAs may be any guide RNA that functions with a Cas endonuclease. Individual guide RNAs may include a separate crRNA molecule and tracrRNA molecule, or individual guide RNAs may be single molecules that include both crRNA and tracrRNA sequences.

The set of Cas complexes may include a single Cas endonuclease and multiple pairs of guide RNAs.

Protection of the ends of the target nucleic acid may include the binding of the Cas complexes to one or both ends. The Cas complexes that bind to the ends of the target nucleic acid may be catalytically inactive.

Protection of the ends of the target nucleic acid may include cleavage of the target nucleic acids at one or both ends.

Degradation of unprotected nucleic acids may include digestion with an exonuclease, such as exonuclease I, exonuclease II, exonuclease III, exonuclease IV, exonuclease V, exonuclease VI, exonuclease VII, or exonuclease VIII.

The target nucleic acid may be detected by any suitable means, such as by hybridization, spectrophotometry, sequencing, electrophoresis, amplification, fluorescence detection, or chromatography.

In another aspect, the invention provides methods of detecting a nucleic acid. The methods include exposing a population of nucleic acids containing a nucleic acid of interest to a set of complexes, each of which contains a Cas endonuclease and a guide RNA that targets a sequence absent from the nucleic acid of interest; digesting the targeted nucleic acids using the Cas endonuclease-guide RNA complexes; and detecting the nucleic acid of interest. The population of nucleic acids may come from any source. For example, the source may be an organism, such as a human, non-human animal, plant, or other type of organism. The source may be a tissue sample from an animal, such as blood, serum, plasma, skin, conjunctiva, gastrointestinal tract, respiratory tract, vagina, placenta, uterus, oral cavity or nasal cavity. The source may be an environmental source, such as a soil sample or water sample, or a food source, such as a food sample or beverage sample. The nucleic acids may be isolated, purified, or partially purified from a source. Alternatively, nucleic acids may be contained in sample that has not been processed.

The nucleic acid of interest may be from the genome of a pathogen, such as a virus, bacterium, or fungus. The nucleic acids may come from an organism, and the nucleic acid of interest may be foreign to the genome of the organism. For example, the nucleic acid of interest may be from a pathogen of the organism. The nucleic acid of interest may be from a virus that infects the organism from which the nucleic acids are obtained. The nucleic acid of interest may be a viral nucleic acid that has integrated into the genome of the host organism. Additionally or alternatively, the nucleic acid of interest may be a viral nucleic acid that exists separately from the nucleic acids of the host organism. The nucleic acids may come from an organism, and the nucleic acid of interest may be native to the organism. For example, the nucleic acid of interest may be from the nuclear genome, mitochondrial genome, or chloroplast genome of the organism.

The nucleic acid of interest may have a particular size. For example, the nucleic acid of interest may be between 100 and 10,000 nucleotides in length, or it may be greater than 10,000 nucleotides. The nucleic acid of interest may be larger than any remaining nucleic acids after digestion. Thus, the difference in size between the nucleic acid of interest and the nucleic acid fragments after digestion may facilitate detection of the nucleic acid of interest.

The complexes may include any Cas endonuclease. For example, the Cas endonuclease may be Cas9, Cas13 Cpf1, C2c1, C2c3, C2c2, CasX, or CasY, including modified versions of Cas9, Cas13, Cpf1, C2c1, C2c3, C2c2, CasX, or CasY in which the amino acid sequence has been altered.

The guide RNAs may be any guide RNA that functions with a Cas endonuclease. Individual guide RNAs may include a separate crRNA molecule and tracrRNA molecule, or individual guide RNAs may be single molecules that include both crRNA and tracrRNA sequences.

The set of complexes may include a single Cas endonuclease and a set of guide RNAs. The set may include at least 1000 different complexes.

Digestion of the targeted nucleic acids may cleave the targeted nucleic acids to molecules of a certain size. For example, the digested nucleic acids may be less than about 5000 nucleotides. Digested nucleic acids may be smaller than the nucleic acid of interest, thereby facilitating detection of the nucleic acid of interest.

The nucleic acid of interest may be detected by any suitable means, such as by hybridization, spectrophotometry, sequencing, electrophoresis, amplification, fluorescence detection, or chromatography.

In another embodiment, the invention provides methods of detecting a nucleic acid among a population of nucleic acids by selective enrichment. In selective enrichment, at least two Cas endonuclease complexes are added to a sample comprising nucleic acid. The Cas endonuclease complexes attach to a target nucleic acid to protect the target in the nucleic acid sample. In some embodiments, more than two Cas endonuclease complexes are added to the sample to protect the target area of interest. For example, a target nucleic acid is protected by introducing a first Cas endonuclease/guide RNA complex and a second Cas endonuclease/guide RNA complex that binds to the target nucleic acid to the sample.

At least a portion of unprotected nucleic acid is digested. For example, one or more exonucleases may be introduced that promiscuously digest unbound, unprotected nucleic acid. While the exonucleases act, the segment containing the target nucleic acid of interest is protected by the bound complexes and survives the digestion step intact. The exonuclease may be deactivated after a prescribed time period that allows for at least some of the unprotected nucleic acid to be digested or degraded. The isolated target can be removed from Cas by known laboratory techniques, including heating, chemical denaturation, sonic, or any suitable method, including wash steps.

Methods of the invention further comprise adding linkers to the sample. Linkers are added to the sample and attach to the ends of the target nucleic acid. The added linkers only attach to the target nucleic acid ends and do not attach to the unprotected nucleic acid because the unprotected nucleic acid was degraded by the exonuclease. For example, the linkers may attach by ligation, hybridization, or annealing. The linkers are used to ligate to polynucleotides or to connect to any other molecular construct, such as to a solid substrate or bead. For example, linkers are attached by known laboratory methods, such as linker ligation procedures using ligase enzymes. Linkers act as the substrate for detection steps. Linkers can be double-stranded, single-stranded, partially double-stranded or single-stranded, or an Illumina Y-adaptor. Methods of the invention may include a wash step for purification before adding or attaching linkers, after adding or attaching linkers, or at any other time. For example, wash steps may include a wash on a column, a bead wash, and isolation or purification such as gel purification, e.g., by SDS-PAGE.

Because methods of the invention work to capture very long (500, 1,000, 5,000 bases) targets, the methods are useful as sample preparation for sequencing technologies that can sequence very long nucleic acid fragments. For example, third generation sequencing technologies that offer long reads or can sequence long nucleic acid molecules. For example, Oxford Nanopore provides nanopore sequencing products for the direct, electronic analysis of single molecules.

The method includes detecting the target nucleic acid segment. Any suitable technique may be used to detect the target nucleic acid segment. For example, detection may be performed using DNA staining, spectrophotometry, sequencing, fluorescent probe hybridization, fluorescence resonance energy transfer, optical microscopy, electron microscopy, others, or combinations thereof. Detecting the target nucleic acid segment indicates the presence of the mutation in the subject (i.e., a patient), and a report may be provided describing the mutation in the patient.

The Cas endonuclease complexes may be different from each other, or they may be the same. Preferably, the Cas complexes are Cas9 complexes. The Cas complexes include a Cas endonuclease and a guide RNA. The Cas endonuclease may include any Cas endonuclease. For example, the Cas endonuclease may be Cas9, Cas13, Cpf1, C2c1, C2c3, C2c2, CasX, or CasY, including modified versions of Cas9, Cas13, Cpf1, C2c1, C2c3, C2c2, CasX, or CasY in which the amino acid sequence has been altered. The Cas endonuclease is catalytically inactive. For example, the Cas endonuclease may be Streptococcus pyogenes Cas9 that has a D10A and/or a R1335K mutation, Acidaminococcus sp. BV3L6 Cpf1 that has a D908 mutation, or Lachnospiraceae bacterium ND2006 that has a D832 mutation.

The guide RNAs may be any guide RNA that functions with a Cas endonuclease. Individual guide RNAs may include a separate crRNA molecule and tracrRNA molecule, or individual guide RNAs may be single molecules that include both crRNA and tracrRNA sequences.

Degradation of unprotected nucleic acids may include digestion with an exonuclease, such as exonuclease I, exonuclease II, exonuclease III, exonuclease IV, exonuclease V, exonuclease VI, exonuclease VII, or exonuclease VIII. In certain embodiments of the invention, the exonuclease is deactivated after a portion of the nucleic acid is digested. If left to completion, the exonuclease would digest all, or nearly all, of the unprotected nucleic acid. In some instances, heat is used to deactivate the exonuclease so that the exonuclease stops digesting non-target nucleic acid in the sample.

In some embodiments of the invention, multiple binding proteins (e.g., Cas endonuclease complexes) are used to protect a target nucleic acid in, or prepared from, a biological sample. Methods include binding proteins to ends of the target nucleic acid and binding one or more additional proteins along the interstitial length of the target nucleic acid. The proteins that bind to the target nucleic acid may be any proteins that bind a nucleic acid in a sequence-specific manner. Some or all of the proteins may be the same, or each protein may be different. Preferably, the proteins that bind to the ends of target nucleic acid are the same. In some embodiments, all of the proteins, i.e., the proteins that bind the ends and the proteins that bind internal regions, are the same.

The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). A preferred protein for end binding is a Cas endonuclease. Cas is complexed with target nucleic acid using guide RNAs that are designed for sequence-specific binding. Proteins for binding between the ends may also be Cas proteins or others that protect sequence from degradation. An ideal protein is catalytically-inactive (dead) Cas (dCas). Whatever binding protein modality is used, the intermediate regions (i.e., between the ends) must be tiled along the sequence at spaced intervals designed to decrease degradation of the target sequence.

Methods of the invention are useful in a wide variety of applications. For example, because methods of the invention preserve target sequence, they are ideal for detection of sequence that is present in a sample at low abundance. Thus, methods of the invention are useful for analysis of cfDNA in blood or blood products (e.g., plasma). As a result, methods of the invention allow the early detection of genomic alterations indicative of cancer and identification of genetic disorders of a fetus in utero. Methods of the invention allow detection of genomic alterations, big and small, such as duplications, translocations, loss of heterozyosity (LOH), inversions and the like.

### BRIEF DESCRIPTION OF THE DRAWINGS

FIG. 1 diagrams a method of detecting a nucleic acid in a second embodiment.

FIG. 2 illustrates the second embodiment of the invention.

FIG. 3 shows a kit of the invention.

FIG. 4 diagrams a method of detecting a nucleic acid in a first embodiment.

FIG. 5 illustrates a method of detecting a nucleic acid in the first embodiment.

FIG. 6 illustrates a method of detecting a nucleic acid in a third embodiment.

FIG. 7 illustrates a method of detecting a nucleic acid in the third embodiment.

### DETAILED DESCRIPTION

Provided herein are methods of detecting nucleic acids. The nucleic acid of interest may be detected by first using Cas endonuclease to degrade substantially all nucleic acid in a sample except for the nucleic acid of interest, then detected the presence of the nucleic acid of interest. In related methods, Cas endonuclease complexes are used to protect the nucleic acid of interest while unprotected nucleic acid is digested, e.g., by exonuclease, followed by detecting the nucleic acid of interest that remains. The invention provides methods of detecting a nucleic acid of interest in a population of nucleic acids by eliminating all of the nucleic acids other than the one of interest. Because the methods of the invention do not require “fishing” target nucleic acids from a population, they avoid problems of target size, sensitivity, and target adulteration associated with methods that rely on hybrid capture or PCR amplification. In addition, the methods of the invention allow detection of multiple nucleic acids of interest in a single assay.

The aforementioned advantages make the methods of the invention useful for a variety of applications. For example, the methods can be used to detect foreign nucleic acids in a host organism. Thus, they allow detection of infectious agents, such as viruses, bacteria, and fungi, in humans, other animals, and plants. In particular, pathogenic microbes can be detected. Alternatively, they permit detection of low-abundance nucleic acids that are native to an organism, such as genes from a mitochondrial or chloroplast genome or nuclear genes that are present in only a minority of cells in a sample. The methods can also be used to detect nucleic acids from a microbe, and thus the microbe itself, in a sample from an environmental source, such as a soil or water, or from a food source. In addition, because multiple nucleic acids can be simultaneously detected, the methods of the invention are useful for determining the microbiome of a sample, such as bodily tissue or an external source.

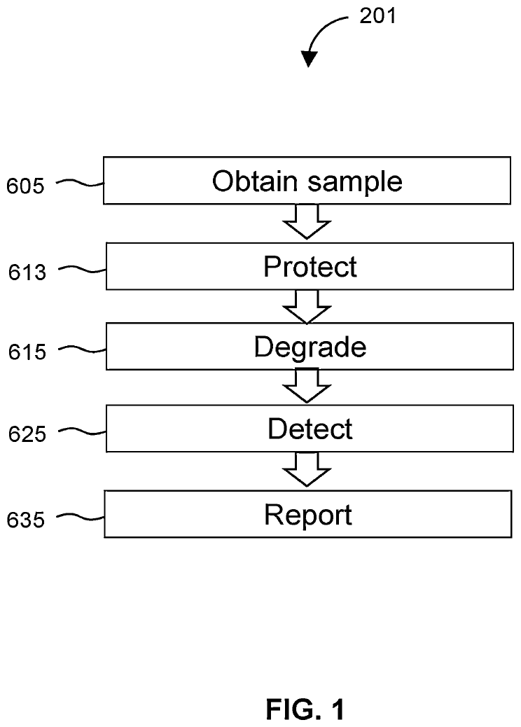

FIG. 1 diagrams a method 201 of detecting a nucleic acid. The method 201 includes obtaining 605 a sample. The method includes protecting 613, in a population of nucleic acids, first and/or second ends of a target nucleic acid using respective first and/or second binding proteins such as Cas endonuclease (e.g., complexed with a guide RNA). The method 201 further includes degrading 615 unprotected nucleic acids and detecting 625 the protected nucleic acid. Preferably, the detected nucleic acid is reported 635 as being present in the sample.

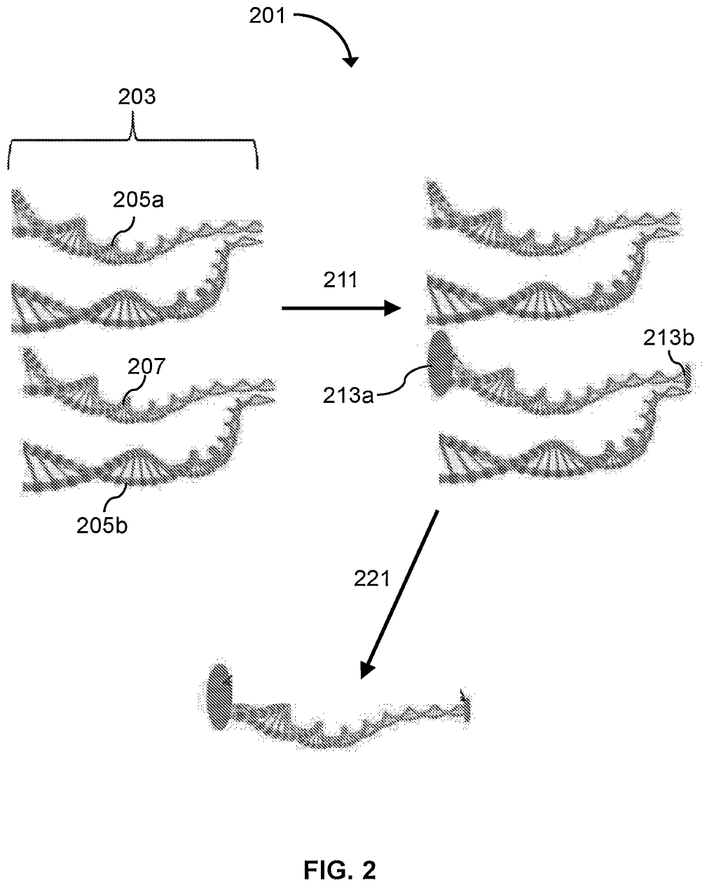

FIG. 2 illustrates the method 201. A population 203 of nucleic acids 205a, 205b, including a target nucleic acid 207, is provided. The target nucleic acid 207 is protected 211 by allowing Cas complexes 213a, 213b to bind to sequences at the ends of the target nucleic acid 207. The target nucleic acid 207 may be a portion of larger nucleic acid molecule, and the ends of the target nucleic acid 207 may not be the ends of a nucleic acid molecule, i.e., the ends may not be free 5′ phosphate groups or free 3′ OH groups. Binding of the Cas complexes to the ends of the target nucleic provides protection against exonuclease digestion. Nucleic acids 205a, 205b in the population 203 are then degraded 221, but the target nucleic acid 207 is protected from degradation. Preferably, degradation occurs via exonuclease digestion. The target nucleic acid 207 may then be detected by any suitable means.

The nucleic acids may come from any source, as described elsewhere herein. Also, as described elsewhere herein, the nucleic acids may have been isolated, purified, or partially purified, or the samples may not have been processed. The target nucleic acid may be any nucleic acid of interest of any size, as described elsewhere herein.

The methods are also useful for detecting nucleic acid from an infectious agent, such as a virus, as may present in a host. Methods are addressed to challenges by which viral nucleic acid may be difficult to detect among abundant host DNA. Thus the detected nucleic acid may be of an infecting virus. Obtaining the sample may include taking a tissue sample from a patient and extracting or accessing DNA therein. The DNA of an infecting virus is isolated by digesting away substantial amounts of non-viral DNA. E.g., using method 101, a plurality of guide RNAs specific to a human genome (but having no match in the viral genome) is used to digest away the host genetic material, leaving only viral DNA present, such that detecting the viral DNA confirms the presence of the virus in the patient. Preferably, method 201 is used and the viral DNA is protected using binding proteins (e.g., Cas endonuclease) while unprotected nucleic acid is ablated (using, e.g., exonuclease). The detected viral DNA may be of any suitable virus including retroviruses that integrate into the host genome and virus present as viral episomes.

Thus in a preferred embodiment, the method includes providing guide RNAs that are specific to a viral genome, such as HIV, and using those guide RNAs with Cas endonuclease to protect a fragment of viral DNA in a sample from a patient. After digesting away unprotected DNA, the remaining DNA is detected, confirming the presence of the virus in the host genome. The methods thus provides a rapid and reliable viral test, that can detect retroviral proviral DNA and/or viral episomes, and thus detect viral infections at any stage.

The Cas complexes may include any Cas endonuclease, as described elsewhere herein. The Cas endonuclease may be catalytically inactive. For example and without limitation, the Cas endonuclease may be Streptococcus pyogenes Cas9 that has a D10A and/or R1335K mutation, Acidaminococcus sp. BV3L6 Cpf1 that has a D908 mutation, or Lachnospiraceae bacterium ND2006 that has a D832 mutation.

The Cas complexes that bind the ends of the target nucleic acid may be different from each other, or they may be the same. Preferably, the Cas complexes that bind the ends of the target nucleic acid have the same Cas endonuclease complexed with different guide RNAs, with each guide containing a sequence that targets one end of the target nucleic acid.

The guide RNAs may be single-molecule guides or two-molecule guides, as described elsewhere herein.

Degradation of unprotected nucleic acids may occur by any suitable means. Preferably, unprotected nucleic acids are degraded by digestion with an exonuclease, such as exonuclease I, exonuclease II, exonuclease III, exonuclease IV, exonuclease V, exonuclease VI, exonuclease VII, or exonuclease VIII. Digestion may destroy all or substantially all nucleic acids in the population other than the target. For example, at least 90%, at least 95%, at least 98%, at least 99%, at least 99.9% of targeted nucleic acids may be digested. Digestion may degrade nucleic acids to individual nucleotides or to small fragments that are distinguishable from the intact target. For example, after degradation, nucleic acids other than the target may have fewer than 20 nucleotides, fewer than 10 nucleotides, fewer than 5 nucleotides, fewer than 4 nucleotides, or fewer than 3 nucleotides.

After digestion, the target nucleic acid may be detected by any suitable means, as described elsewhere herein.

Protection of the target nucleic acid may occur simply by binding of a Cas complex to each end of the target, thereby preventing exonuclease digestion of the target. Alternatively or additionally, protection may involve binding of the Cas complexes or cleavage of at one or both of the binding sites near the end of the target nucleic acid.

In certain embodiments, the method 201 has applications in metagenomics. Metagenomics includes to the study of genetic material recovered directly from environmental samples. While traditional microbiology and microbial genome sequencing and genomics relied upon cultivated clonal cultures, early environmental gene sequencing cloned specific genes (often the 16S rRNA gene) to produce a profile of diversity in a natural sample. Here, methods of the invention are useful to take essentially unbiased samples of all genes from all the members of the sampled communities. Because of their ability to reveal the previously hidden diversity of microscopic life, methods of the disclosure applied to metagenomics offers novel views of the microbial world. Specifically, using a mixture of guide RNAs, the method 201 may be used to isolate a plurality of representative sample fragments of microbial DNA from an environmental sample, such that the set of sample fragments are a representative sample of microbial diversity in the environmental sample. In some embodiments, the methods 201 are performed with an abundance of pseudo-random guide RNAs in Cas endonuclease complexes. Pairs of the complexes isolate DNA fragments from an environmental sample. The collected set of fragments is essentially a vertical slice through the microbial genetic information in the sample, and thus is representative of microbial diversity therein. Those fragments may be analyzed to reveal microbial diversity in the sample, even without culturing individual microbes or knowing a priori the species that may be present in the sample. The fragments may be analyze by, for example, sequencing. Since methods of the invention are useful to isolate long, intact nucleic acid molecules, the methods are particularly useful for long-read, single-molecule sequencing platforms such as those of Oxford Nanopore or Pacific Biosciences. Using such a sequencing platform with a method 201, one may perform a metagenomic survey of a sample and microbial ecology may thus be investigated at a much greater scale and detail than before.

Methods of the invention can be used to identify the microbiome, i.e., the collective genetic material of the microbiota. The microbiota is the ecological community of commensal, symbiotic and pathogenic microorganism found in and on all multicellular organisms studied to date. A microbiota includes bacteria, archaea, protists, fungi and viruses. Microbiota have been found to be crucial for immunologic, hormonal, and metabolic homeostasis of their host. The symbiotic relationship between a host and its microbiota shapes the immune system of mammalians, insects, plants, and aquatic organisms.

Embodiments of the invention include identification of a microbiome from a sample. Such embodiments include a method 101 or method 201 of detecting a nucleic acid of interest. The methods may include detecting multiple microbes in a sample. Preferably, methods include protecting one or both ends of a nucleic acid from a genome of a microbe in a sample using a first Cas9 complex and a second Cas9 complex, degrading unprotected nucleic acids, and detecting at least one protected nucleic acid, thereby detecting the microbe in the sample. Multiple nucleic acids from multiple microbes can be detected using sets of pairs of Cas9 complexes, allowing the microbiome of a sample to be determined. The method may include reporting the microbiome of a sample.

Kits and methods of the invention are useful with methods disclosed in U.S. Provisional Patent Application 62/526,091, filed Jun. 28, 2017, for POLYNUCLEIC ACID MOLECULE ENRICHMENT METHODOLOGIES and U.S. Provisional Patent Application 62/519,051, filed Jun. 13, 2017, for POLYNUCLEIC ACID MOLECULE ENRICHMENT METHODOLOGIES, both incorporated by reference.

The method 201 uses a double-protection to select one or both ends of DNA segments. Unprotected segments are digested and the remaining molecules are either counted or sequenced. The method 201 is well suited for the analysis of small portions of DNA, degraded samples, samples in which the target of interest is extremely rare, and particularly for environmental samples, e.g., for pathogen detection or metagenomics.

The method 201 includes a negative enrichment step that leaves the target of interest intact and isolated as a segment of DNA. The methods are useful for the isolation of intact DNA fragments of any arbitrary length and may preferably be used in some embodiments to isolate (or enrich for) arbitrarily long fragments of DNA, e.g., tens, hundreds, thousands, or tens of thousands of bases in length or longer. Long, isolated, intact fragments of DNA may be analyzed by any suitable method such as simple detection (e.g., via staining with ethidium bromide) or by single-molecule sequencing. Embodiments of the invention provide kits that may be used in performing methods described herein.

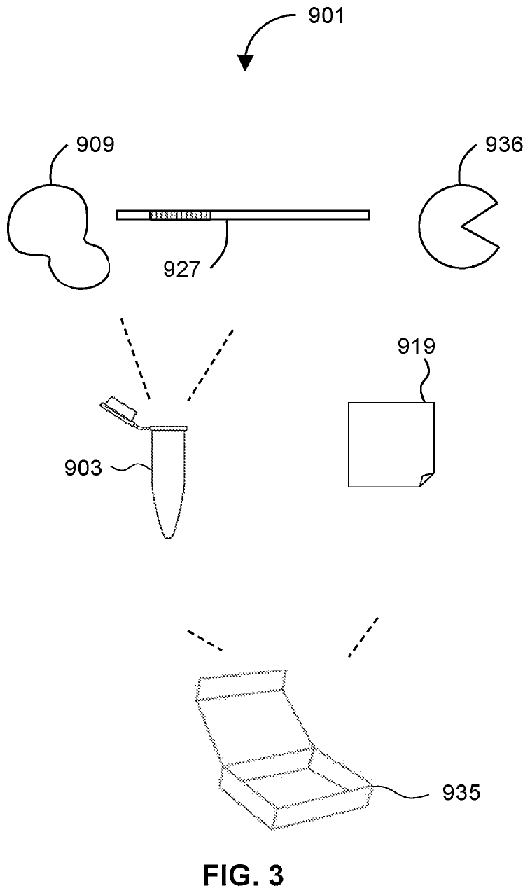

FIG. 3 shows a kit 901 of the invention. The kit 901 may include reagents 903 for performing the steps described herein. For example, the reagents 903 may include one or more of a Cas endonuclease 909, a guide RNA 927, and exonuclease 936. The kit 901 may also include instructions 919 or other materials. The reagents 903, instructions 919, and any other useful materials may be packaged in a suitable container 935. Kits of the invention may be made to order. For example, an investigator may use, e.g., an online tool to design guide RNA and reagents for the performance of methods 101, 201. The guide RNAs 927 may be synthesized using a suitable synthesis instrument. The synthesis instrument may be used to synthesize oligonucleotides such as gRNAs or single-guide RNAs (sgRNAs). Any suitable instrument or chemistry may be used to synthesize a gRNA. In some embodiments, the synthesis instrument is the MerMade 4 DNA/RNA synthesizer from Bioautomation (Irving, TX). Such an instrument can synthesize up to 12 different oligonucleotides simultaneously using either 50, 200, or 1,000 nanomole prepacked columns. The synthesis instrument can prepare a large number of guide RNAs 927 per run. These molecules (e.g., oligos) can be made using individual prepacked columns (e.g., arrayed in groups of 96) or well-plates. The resultant reagents 903 (e.g., guide RNAs 917, endonuclease(s) 909, exonucleases 936) can be packaged in a container 935 for shipping as a kit.

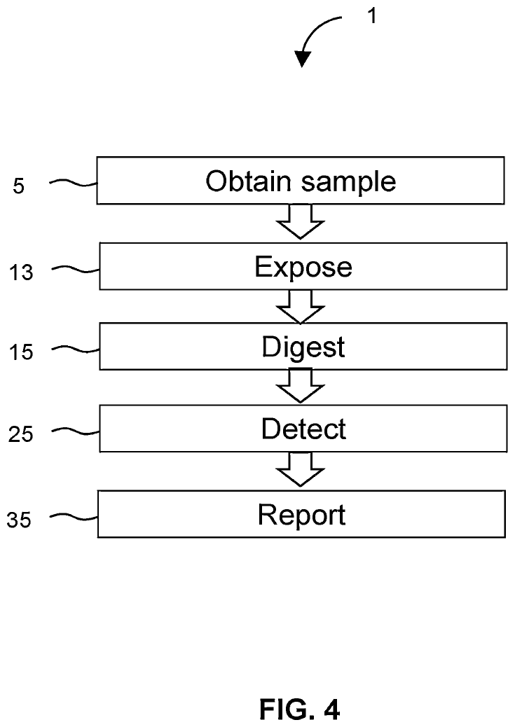

The invention also provides an alternative method of detecting a nucleic acid of interest. FIG. 4 diagrams a method 101 of detecting a nucleic acid. The method 101 includes obtaining 5 a sample and exposing 13 a population of nucleic acids comprising a nucleic acid of interest to a plurality of complexes. Each complex includes a Cas endonuclease and a guide RNA that targets a sequence absent from the nucleic acid of interest. The method 101 includes digesting 15 nucleic acids targeted by the plurality of complexes using the plurality of complexes and detecting 25 the nucleic acid of interest. The method 101 may optionally include reporting 35 the nucleic acid of interest in the sample.

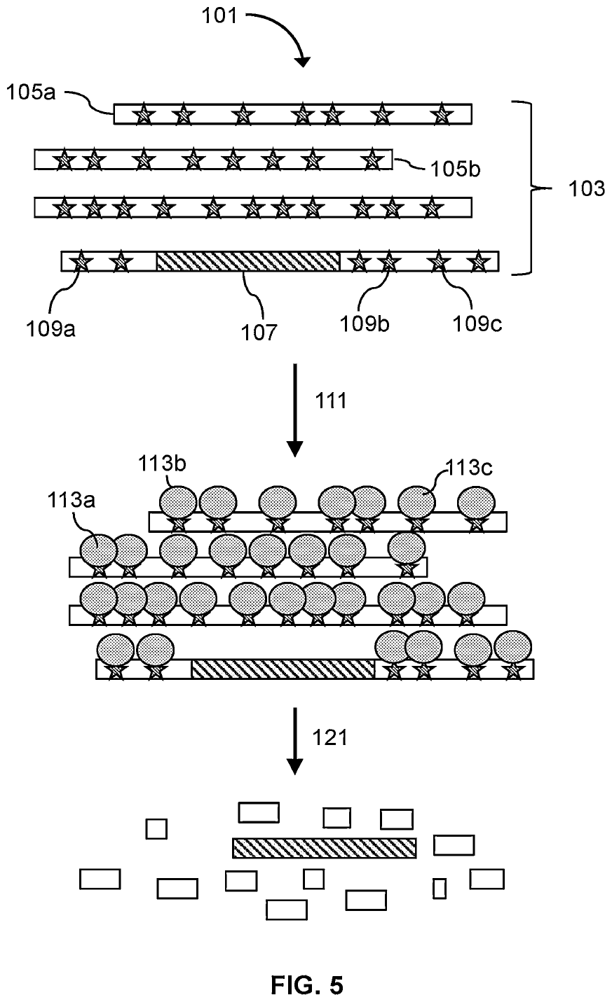

FIG. 5 illustrates the method 101. A population 103 of nucleic acids 105a, 105b, including a nucleic acid of interest 107, is provided. The nucleic acids 105a, 105b include numerous target sequences 109a, 109b, and 109c for a set of Cas complexes 113a, 113b, 113c, but the nucleic acid of interest does not contain a target sequence. The population 103 is exposed 111 to the set of Cas complexes 113a, 113b, and 113c, which are targeted to the various target sequences 109a, 109b, and 109c. The nucleic acids 105a, 105b are then digested 121 by the Cas complexes 113a, 113b, and 113c. Most nucleic acids 105a, 105b are digested into small fragments, but the nucleic acid of interest 107, which was not targeted by a Cas complex, remains intact. The nucleic acid of interest 107 may then be detected by any suitable means.

The Cas complexes include a Cas endonuclease and a guide RNA. For example, the Cas endonuclease may be Cas9, Cas13 Cpf1, C2c1, C2c3, C2c2, CasX, or CasY, including modified versions of Cas9, Cas13, Cpf1, C2c1, C2c3, C2c2, CasX, or CasY in which the amino acid sequence has been altered. Preferably, the Cas endonuclease is Cas9. The Cas endonuclease may be from any bacterial species. For example and without limitation, the Cas endonuclease may be from Bacteroides coprophilus, Campylobacter jejuni susp. jejuni, Campylobacter lari, Fancisella novicida, Filifactor alocis, Flavobacterium columnare, Fluviicola taffensis, Gluconacetobacter diazotrophicus, Lactobacillus farciminis, Lactobacillus johnsonii (e), Legionella pneumophila, Mycoplasma gallisepticum, Mycoplasma mobile, Neisseria cinerea, Neisseria meningitidis, Nitratifractor salsuginis, Parvibaculum lavamentivorans, Pasteurella multocida, Sphaerochaeta globusa, Streptococcus pasteurianus, Streptococcus thermophilus, Sutterella wadsworthensis, and Treponema denticola.

A guide RNA mediates binding of the Cas complex to the guide RNA target site via a sequence complementary to a sequence in the target site. Typically, guide RNAs that exist as single RNA species comprise a CRISPR (cr) domain that is complementary to a target nucleic acid and a tracr domain that binds a CRISPR/Cas protein. However, guide RNAs may contain these domains on separate RNA molecules.

Typically, the set of Cas complexes includes a single Cas endonuclease and a panel of guide RNAs that have common tracr sequences and different targeting sequences. The panel of targeting sequences includes sequences complementary to as many regions in the population of nucleic acids as possible without targeting the nucleic acid of interest. For example, if the population of nucleic acids is from a host organism and the nucleic acid of interest is from a microbial pathogen of that host, the panel of guide RNAs may be designed to target sites throughout the host genome without targeting a sequence from the genome of the microbial pathogen. For example, the panel of guide RNAs may include at least 100, at least 1000, at least 10,000, at least 100,000 at least 1,000,000, or at least 10,000,000 different species. Thus, when the guide RNAs from the panel are complexed with the Cas endonuclease, the set of complexes may include at least 100, at least 1000, at least 10,000, at least 100,000 at least 1,000,000, or at least 10,000,000 different complexes.

The population of nucleic acids may come from any source. The source may be an organism, such as a human, non-human animal, plant, or other type of organism. The source may be a tissue sample from an animal, such as blood, serum, plasma, skin, conjunctiva, gastrointestinal tract, respiratory tract, vagina, placenta, uterus, oral cavity or nasal cavity. The source may be an environmental source, such as a soil sample or water sample, or a food source, such as a food sample or beverage sample.

The population of nucleic acids may have been isolated, purified, or partially purified from a source. Techniques for preparing nucleic acids from tissue samples and other sources are known in the art and described, for example, in Green and Sambrook, Molecular Cloning: A Laboratory Manual (Fourth Edition), Cold Spring Harbor Laboratory Press, Woodbury, NY 2,028 pages (2012), incorporated herein by reference. Alternatively, the nucleic acids may be contained in sample that has not been processed. The nucleic acids may single-stranded or double-stranded. Double-stranded nucleic acids may be DNA, RNA, or DNA/RNA hybrids. Preferably, the nucleic acids are double-stranded DNA.

The nucleic acid of interest may be from the genome of a pathogen, such as a virus, bacterium, or fungus. The population of nucleic acids may come from an organism, and the nucleic acid of interest may be foreign to the genome of the organism. For example, the nucleic acid of interest may be from a pathogen of the organism. The nucleic acid of interest may be from a virus that infects the organism from which the nucleic acids are obtained. The nucleic acid of interest may be a viral nucleic acid that has integrated into the genome of the host organism. Additionally or alternatively, the nucleic acid of interest may be a viral nucleic acid that exists separately from the nucleic acids of the host organism. The nucleic acid of interest may be native to the organism from which the population has been obtained. For example, the nucleic acid of interest may be from the nuclear genome, mitochondrial genome, or chloroplast genome of the organism. The population of nucleic acids may come from a tissue sample from an organism, and the nucleic acid of interest may be a nucleic acid that is present in that tissue in a low abundance and/or is indicative of a pathological or medical condition.

The nucleic acid of interest may have a particular size. For example, the nucleic acid of interest may be between 100 and 10,000 nucleotides in length, or it may be greater than 1000 nucleotides in length.

Digestion of the targeted nucleic acids may cleave the targeted nucleic acids to molecules of a certain size. For example, the digested nucleic acids may be less than about 10 nucleotides, less than about 20 nucleotides, less than about 50 nucleotides, less than about 100 nucleotides, less than about 200 nucleotides, less than about 500 nucleotides, less than about 1000 nucleotides, less than about 2000 nucleotides, or less than about 5000 nucleotides. Digested nucleic acids may be smaller than the nucleic acid of interest. All or substantially all targeted nucleic acids may be digested. For example, at least 90%, at least 95%, at least 98%, at least 99%, at least 99.9% of targeted nucleic acids may be digested.

The nucleic acid of interest may be detected by any suitable means. Methods of detection of nucleic acids are known in the art and described, for example, in Green and Sambrook, Molecular Cloning: A Laboratory Manual (Fourth Edition), Cold Spring Harbor Laboratory Press, Woodbury, NY 2,028 pages (2012), incorporated herein by reference. For example and without limitation, nucleic acid of interest may be detected by hybridization, spectrophotometry, sequencing, electrophoresis, amplification, fluorescence detection, or chromatography. Detection may be based on difference in size between the nucleic acid of interest and the fragments of other nucleic acids that remain after digestion. For example, after digestion, fragments of targeted nucleic acids may fall below a threshold size, while the nucleic acid of interest may exceed the threshold size. For example, after digestion, the nucleic acid of interest may be the only nucleic acid greater than about 10 nucleotides, greater than about 20 nucleotides, greater than about 50 nucleotides, greater than about 100 nucleotides, greater than about 200 nucleotides, greater than about 500 nucleotides, greater than about 1000 nucleotides, greater than about 2000 nucleotides, or greater than about 5000 nucleotides.

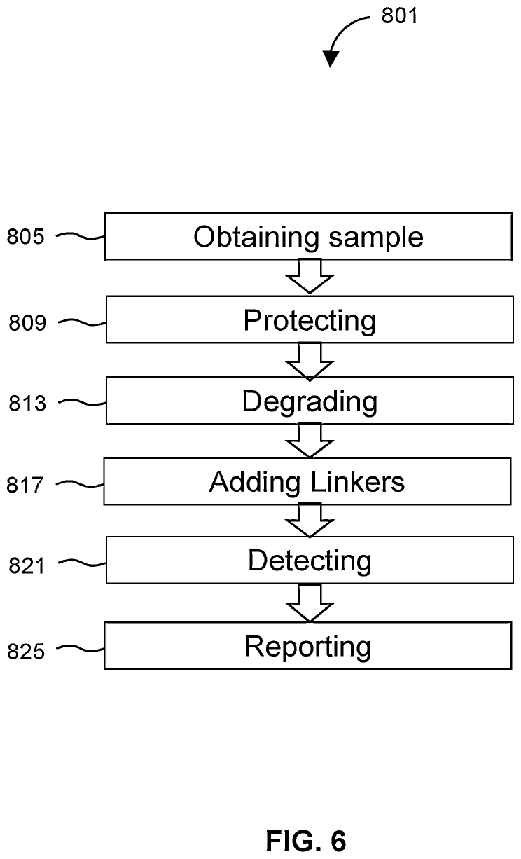

FIG. 6 diagrams a method 801 of detecting a nucleic acid using selective enrichment. The method 801 includes obtaining 805 a sample comprising a plurality of nucleic acid. The nucleic acids may come from any source, as described elsewhere herein. Also, as described elsewhere herein, the nucleic acids may have been isolated, purified, or partially purified, or the samples may not have been processed. The target nucleic acid may be any nucleic acid of interest of any size, as described elsewhere herein. A sample comprises a population of nucleic acids, including a target nucleic acid.

Cas endonuclease complexes are added to the sample to protect 809 target nucleic acid in the sample. Each complex includes a Cas endonuclease and a guide RNA that targets a sequence from the nucleic acid of interest. The target nucleic acid is protected by allowing Cas complexes to bind to sequences at the ends of the target nucleic acid. Protection of the target nucleic acid may occur simply by binding of a Cas complex to each end of the target, thereby preventing exonuclease digestion of the target. The Cas complexes that bind the ends of the target nucleic acid may be different from each other, or they may be the same. Preferably, the Cas complexes that bind the ends of the target nucleic acid have the same Cas endonuclease complexed with different guide RNAs, with each guide containing a sequence that targets one end of the target nucleic acid. The target nucleic acid may be a portion of larger nucleic acid molecule, and the ends of the target nucleic acid may not be the ends of a nucleic acid molecule, i.e., the ends may not be free 5′ phosphate groups or free 3′ OH groups.

The Cas complexes may include any Cas endonuclease. The Cas endonuclease may be catalytically inactive. For example and without limitation, the Cas endonuclease may be Streptococcus pyogenes Cas9 that has a D10A and/or R1335K mutation, Acidaminococcus sp. BV3L6 Cpf1 that has a D908 mutation, or Lachnospiraceae bacterium ND2006 that has a D832 mutation.

The method 801 includes digesting or degrading 813 unprotected nucleic acids in the sample by introducing an exonuclease. The exonuclease is deactivated after a portion of the unprotected nucleic acid in the sample is degraded or digested. Binding of the Cas complexes to the ends of the target nucleic provides protection against exonuclease digestion. Nucleic acids in the sample population are then degraded, but the target nucleic acid is protected from degradation. Preferably, degradation occurs via exonuclease digestion. Degradation of unprotected nucleic acids may occur by any suitable means. Preferably, unprotected nucleic acids are degraded by digestion with an exonuclease, such as exonuclease I, exonuclease II, exonuclease III, exonuclease IV, exonuclease V, exonuclease VI, exonuclease VII, or exonuclease VIII. Digestion may destroy a portion of the nucleic acids in the population other than the target. For example, digestion may degrade nucleic acids to individual nucleotides or to small fragments that are distinguishable from the intact target.

After a period of time sufficient to degrade at least a portion of the nucleic acid that is not the target of interest, the exonuclease is deactivated. The exonuclease may be deactivated by any suitable means. For example, heat may be used to deactivate the exonuclease. In an embodiment, the exonuclease degradation is an incomplete degradation, which means only a portion of the unprotected nucleic acid is digested.

The Cas endonuclease complexes are removed and 817 linkers are added to the sample. The isolated target can be removed from Cas by known laboratory techniques, including heating, chemical denaturation, sonic, or any suitable method, including wash steps. The linkers hybridize or anneal to the end of the target nucleic acid previously protected by the Cas endonuclease complexes. Linkers are used to ligate to polynucleotide or to connect to any other molecular construct, such as to a solid substrate or bead. The linkers do not hybridize or anneal to the degraded, unprotected nucleic acid. For example, linkers are attached by known laboratory methods, such as linker ligation procedures using ligase enzymes. Linkers act as the substrate for detection steps. Linkers can be double-stranded, single-stranded, partially double-stranded or single-stranded, or an Illumina Y-adaptor. Methods of the invention may include a wash step 750 for purification before adding or attaching linkers, after adding or attaching linkers, or at any other time. For example, wash steps may include a wash on a column, a bead wash, and isolation or purification such as gel purification, e.g., by SDS-PAGE.

Because methods of the invention work to capture very long (500, 1,000, 5,000 bases) targets 707, the methods are useful as sample preparation for sequencing technologies that can sequence very long nucleic acid fragments. For example, third generation sequencing technologies that offer long reads or can sequence long nucleic acid molecules, e.g., sequencing by Oxford Nanopore technologies.

The method 801 includes 821 detecting the target nucleic acid of interest. The target nucleic acid, or nucleic acid of interest, may then be detected by any suitable means. Methods of detection of nucleic acids are known in the art and described, for example, in Green and Sambrook, Molecular Cloning: A Laboratory Manual (Fourth Edition), Cold Spring Harbor Laboratory Press, Woodbury, NY 2,028 pages (2012), incorporated herein by reference. For example and without limitation, nucleic acid of interest may be detected by hybridization, spectrophotometry, sequencing, electrophoresis, amplification, fluorescence detection, or chromatography. Non-limiting examples of detection methods include PCR, hybrid capture, Next Generation Sequencing, and sequencing such as according to Pacific Biosciences, Oxford Nanopore, Helicos Biosciences, and optical sequencing.

The method 801 may optionally include reporting 825 the nucleic acid of interest in the sample. In some embodiments, the method includes reporting that one or more target nucleic acids are present in the sample.

Methods of the invention are useful in a wide variety of applications. For example, because methods of the invention preserve target sequence, they are ideal for detection of sequence that is present in a sample at low abundance. Thus, methods of the invention are useful for analysis of cfDNA in blood or blood products (e.g., plasma). As a result, methods of the invention allow the early detection of genomic alterations indicative of cancer and identification of genetic disorders of a fetus in utero. Methods of the invention allow detection of genomic alterations, big and small, such as duplications, translocations, loss of heterozyosity (LOH), inversions and the like.

In some embodiments of the invention, multiple binding proteins are used to protect a target nucleic acid in, or prepared from, a biological sample. Methods include binding proteins to ends of the target nucleic acid and binding one or more additional proteins along the interstitial length of the target nucleic acid. The proteins that bind to the target nucleic acid may be any proteins that bind a nucleic acid in a sequence-specific manner. Some or all of the proteins may be the same, or each protein may be different. Preferably, the proteins that bind to the ends of target nucleic acid are the same. In some embodiments, all of the proteins, i.e., the proteins that bind the ends and the proteins that bind internal regions, are the same.

The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). Programmable nucleases and their uses are described in, for example, Zhang F, Wen Y, Guo X (2014). “CRISPR/Cas9 for genome editing: progress, implications and challenges”. Human Molecular Genetics. 23 (R1): R40-6. doi:10.1093/hmg/ddu125; Ledford H (March 2016). “CRISPR: gene editing is just the beginning”. Nature. 531 (7593):156-9. doi:10.1038/531156a; Hsu P D, Lander E S, Zhang F (June 2014). “Development and applications of CRISPR-Cas9 for genome engineering”. Cell. 157 (6): 1262-78. doi:10.1016/j.cell.2014.05.010; Boch J (February 2011). “TALEs of genome targeting”. Nature Biotechnology. 29 (2): 135-6. doi:10.1038/nbt.1767; Wood A J, Lo T W, Zeitler B, Pickle C S, Ralston E J, Lee A H, Amora R, Miller J C, Leung E, Meng X, Zhang L, Rebar E J, Gregory P D, Urnov F D, Meyer B J (July 2011). “Targeted genome editing across species using ZFNs and TALENs”. Science. 333 (6040): 307. doi:10.1126/science.1207773; Carroll, D (2011). “Genome engineering with zinc-finger nucleases”. Genetics Society of America. 188 (4): 773-782. doi:10.1534/genetics.111.131433; Urnov, F. D., Rebar, E. J., Holmes, M. C., Zhang, H. S., & Gregory, P. D. (2010). “Genome Editing with Engineered Zinc Finger Nucleases”. Nature Reviews Genetics. 11 (9): 636-646. doi:10.1038/nrg2842, the contents of each of which are incorporated herein by reference.

In a preferred embodiment, the binding proteins are provided by Cas endonucleases. Any suitable Cas endonuclease or homolog thereof may be used. A Cas endonuclease may be Cas9 (e.g., spCas9), catalytically inactive Cas (dCas such as dCas9), Cpf1, C2c2, others, modified variants thereof, and similar proteins or macromolecular complexes. The protein may be a catalytically inactive form of a nuclease, such as a programmable nuclease described above. The protein may be a transcription activator-like effector (TALE). The protein may be complexed with a nucleic acid that guides the protein to an end of the segment. For example, the protein may be a Cas endonuclease in a complex with a guide RNA.

A preferred protein for end binding is a Cas endonuclease. Cas is complexed with target nucleic acid using guide RNAs that are designed for sequence-specific binding. Proteins for binding between the ends may also be Cas proteins or others that protect sequence from degradation. An ideal protein is catalytically-inactive (dead) Cas (dCas). Whatever binding protein modality is used, the intermediate regions (i.e., between the ends) must be tiled along the sequence at spaced intervals designed to decrease degradation of the target sequence.

Methods of the invention further include detecting the target sequence. Binding proteins may be removed prior to detection. The undamaged portion (i.e., that portion that was protected or otherwise not degraded during sample acquisition or sample preparation) of a target may be detected by any means known in the art. For example and without limitation, the intact portion may be detected by DNA staining, spectrophotometry, sequencing, fluorescent probe hybridization, fluorescence resonance energy transfer, optical microscopy, or electron microscopy. Detection methods may include mapping or comparing detected sequence to a reference. Sequence read length depends upon the integrity of the sample obtained. A sequence can be compiled using known bioinformatic methods.

Nucleic acid for analysis may be obtained from any sample type, such as a liquid or body fluid from a subject, such as urine, blood, plasma, serum, sweat, saliva, semen, feces, phlegm, or a liquid biopsy. The sample may be a food sample. The sample may be from an environmental source, such as a soil sample, or water sample.

The nucleic acid of interest may contain a mutation. For example and without limitation, the feature may be an insertion, deletion, substitution, inversion, amplification, duplication, translocation, or polymorphism. The nucleic acid of interest may be from an infectious agent or pathogen. For example, the nucleic acid sample may be obtained from an organism, and the nucleic acid of interest may contain a sequence foreign to the genome of that organism. The nucleic acid of interest may be from a sub-population of nucleic acid within the nucleic acid sample. For example, the nucleic acid of interest may be cell-free DNA, such as cell-free fetal DNA or circulating tumor DNA.

The nucleic acid may be any naturally-occurring or artificial nucleic acid. The nucleic acid may be DNA, RNA, hybrid DNA/RNA, peptide nucleic acid (PNA), morpholino and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

Embodiments of the disclosure involve the isolation or extraction of a nucleic acid from a tissue sample such as, for example, a formalin-fixed, paraffin embedded (FFPE) tissue sample. Such embodiments provide methods that include selectively protecting target nucleic acid in a tissue sample (e.g., an FFPE tissue sample), and extracting the nucleic acid from the tissue sample, to obtain or analyze diagnostically-relevant sequence. Extracting nucleic acid from tissue samples may include binding protein complexes to the ends of a nucleic acid of interest and optionally binding additional binding proteins to the intervening sequence, before or while performing enzymatic proteolysis under optimized conditions, followed by solid-phase extraction of the nucleic acid on glass-fiber or other solid supports. Methods may include binding the proteins along the target nucleic acid, e.g., introducing proteins that will “tile” along a target nucleic acid. Cas endonuclease may be used and it may be preferable to use catalytically inactive Cas endonuclease (“dCas”). The dCas proteins can be introduced along with guide RNAs that target the dCas to intended positions along the nucleic acid. For example, it may be preferable to target dCas to both ends of the target nucleic acid and optionally also to tile the dCas proteins along the nucleic acid, i.e., to bind the dCas proteins at a series of intervening positions between the two ends. So protecting the nucleic acid may be done in conjunction with isolating or extracting the nucleic acid from the tissue sample using any suitable method, such as using an FFPE nucleic acid extraction kit or reagents from a commercial provider. The methods include removing paraffin wax and using a solid phase for final extraction of the nucleic acid.

Replacement of the wax may be done using water through a series of soaks in xylene (or limonene) and various dilutions of ethanol in water. Paraffin wax removal may be done by the direct incubation of a slice of the embedded tissue in proteolytic solution. Final extraction may use a glass-fiber filter in a spin-column format for the solid phase recovery step, magnetic beads, or any other suitable solid phase extraction.

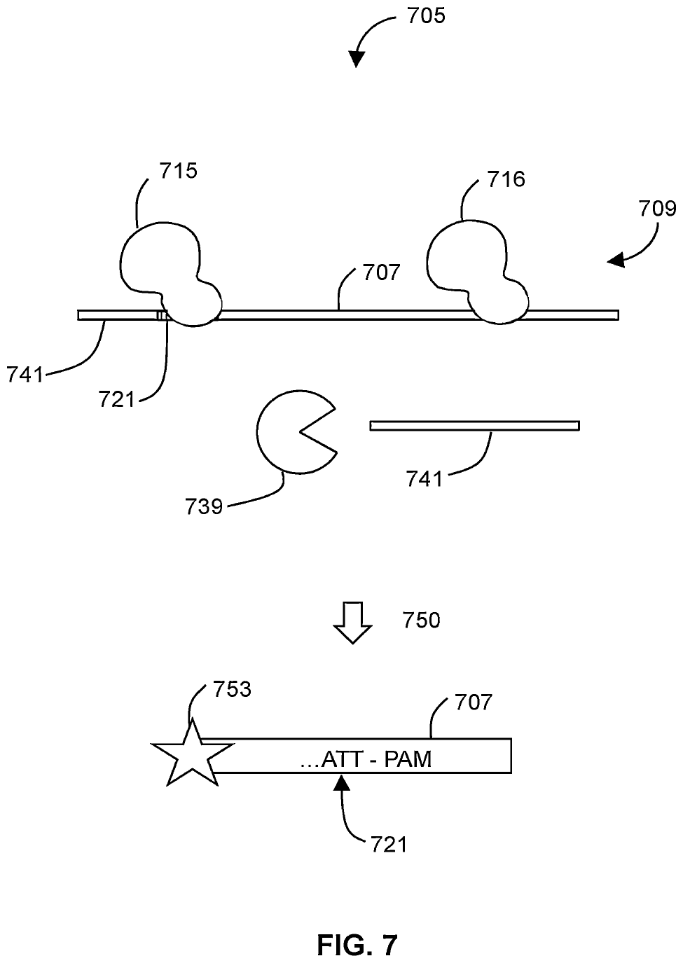

FIG. 7 illustrates operation of the selective enrichment. The sample 705 includes DNA 709 from a subject. The sample 705 is exposed to a first Cas endonuclease/guide RNA complex 715 that binds to a target nucleic acid fragment 707 in a sequence-specific fashion. Specifically, the complex 715 binds to the target sequence 721 in a sequence-specific manner. A segment of the nucleic acid 709, i.e., the target nucleic acid fragment 707, is protected by introducing the first Cas endonuclease/guide RNA complex 715 (that binds to a mutation in the nucleic acid) and a second Cas endonuclease/guide RNA complex 716 that also binds to the nucleic acid. At least a portion of unprotected nucleic acid 741 is digested. For example, one or more exonucleases 739 may be introduced that promiscuously digest unbound, unprotected nucleic acid 741. While the exonucleases 739 act, the segment containing the target nucleic acid of interest, the target nucleic acid fragment 707, is protected by the bound complexes 715, 716 and survives the digestion step intact. The exonuclease 739 may be deactivated after a prescribed time period that allows for at least some of the unprotected nucleic acid 741 to be digested or degraded.

The isolated target 707 can be removed from Cas by known laboratory techniques, including heating, chemical denaturation, sonic, or any suitable method, including wash steps. Linkers may be added to the sample. Linkers 753 are used to ligate to polynucleotides or to connect to any other molecular construct, such as to a solid substrate or bead. For example, linkers are attached by known laboratory methods, such as linker ligation procedures using ligase enzymes. Linkers act as the substrate for detection steps. Linkers can be double-stranded, single-stranded, partially double-stranded or single-stranded, or an Illumina Y-adaptor. Methods of the invention may include a wash step 750 for purification before adding or attaching linkers, after adding or attaching linkers, or at any other time. For example, wash steps may include a wash on a column, a bead wash, and isolation or purification such as gel purification, e.g., by SDS-PAGE.

Because methods of the invention work to capture very long (500, 1,000, 5,000 bases) targets 707, the methods are useful as sample preparation for sequencing technologies that can sequence very long nucleic acid fragments. For example, third generation sequencing technologies that offer long reads or can sequence long nucleic acid molecules. For example, Oxford Nanopore provides nanopore sequencing products for the direct, electronic analysis of single molecules.

The method includes detecting the target nucleic acid segment 707 (which includes the target sequence 721). Any suitable technique may be used to detect the target nucleic acid segment. For example, detection may be performed using DNA staining, spectrophotometry, sequencing, fluorescent probe hybridization, fluorescence resonance energy transfer, optical microscopy, electron microscopy, others, or combinations thereof. Detecting the target nucleic acid segment indicates the presence of the mutation in the subject (i.e., a patient), and a report may be provided describing the mutation in the patient.

A feature of the method is that a specific target nucleic acid, such as a mutation, may be detected by a technique that includes detecting only the presence or absence of a fragment of DNA, and it need not be necessary to sequence DNA from a subject to describe mutations. The method may be performed in fluid partitions, such as in droplets on a microfluidic device, such that each detection step is binary (or “digital”). For example, droplets may pass a light source and photodetector on a microfluidic chip and light may be used to detect the presence of a segment of DNA in each droplet (which segment may or may not be amplified as suited to the particular application circumstance).

The method uses a double-protection to select one or both ends of DNA segments. The gRNA selects for a known mutation on one end. If it doesn't find the mutation, no protection is provided and the molecule gets digested. The remaining molecules are either counted or sequenced. The method is well suited for the analysis of small portions of DNA, degraded samples, samples in which the target of interest is extremely rare, and particularly for the analysis of maternal serum (e.g., for fetal DNA) or a liquid biopsy (e.g., for ctDNA).

The methods are useful for the isolation of intact DNA fragments of any arbitrary length and may preferably be used in some embodiments to isolate (or enrich for) arbitrarily long fragments of DNA, e.g., tens, hundreds, thousands, or tens of thousands of bases in length or longer. Long, isolated, intact fragments of DNA may be analyzed by any suitable method such as simple detection (e.g., via staining with ethidium bromide) or by single-molecule sequencing.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of detecting a nucleic acid, the method comprising:
protecting a target nucleic acid in a sample using at least two Cas endonuclease complexes;
adding an exonuclease to the sample to digest unprotected nucleic acids in the sample, and deactivating the exonuclease after at least a portion of the unprotected nucleic acid is digested;
adding linkers to the sample, wherein the linkers attach to ends of the target nucleic acid; and
detecting the target nucleic acid.

2. The method of claim 1, wherein each Cas endonuclease complex comprises a Cas endonuclease and guide RNA.

3. The method of claim 1, wherein more than two Cas endonuclease complexes are used to protect the target nucleic acid.

4. The method of claim 1, wherein only a portion of the unprotected nucleic acids are degraded.

5. The method of claim 1, wherein the at least one protected nucleic acid comprises the target.

6. The method of claim 1, wherein a first Cas endonuclease complex and a second Cas endonuclease complex are different.

7. The method of claim 6, wherein the first Cas endonuclease complex comprises a first Cas9 protein complexed with a first guide RNA, and the second Cas endonuclease complex comprises a second Cas9 protein complexed with a second guide RNA.

8. The method of claim 7, wherein at least one of the first Cas9 complex and the second Cas9 complex comprises a catalytically inactive Cas9 protein.

9. The method of claim 1, wherein the detecting step comprises one selected from the group consisting of hybridization, spectrophotometry, sequencing, electrophoresis, amplification, fluorescence detection, and chromatography.

10. The method of claim 9, wherein the detecting step comprises sequencing.

11. The method of claim 9, wherein the detecting step comprises polymerase chain reaction (PCR) amplification.

12. The method of claim 1, further comprising a wash step.

13. The method of claim 12, wherein the wash step occurs before adding linkers to the sample.

14. The method of claim 1, wherein the sample is a human sample.

15. The method of claim 14, wherein the sample is urine, blood, plasma, serum, sweat, saliva, semen, feces, phlegm, or a liquid biopsy.

16. The method of claim 1, wherein the sample is a non-human animal sample.

17. The method of claim 1, further comprising tiling a plurality of Cas endonuclease complexes to the target nucleic acid.

18. The method of claim 1, wherein the linkers attach by ligation, hybridization, or annealing.
